(19) United States
(12) Patent Application Publication
(10) Pub. No.: US 2026/0171216 A1
(43) Pub. Date: Jun. 18, 2026

(54) Method for Facilitating Emotionally Aligned Romantic Relationships (Timaci Method)

(71) Applicant: Anna Goodwin, Bradenton, FL (US)

(72) Inventor: Anna Goodwin, Bradenton, FL (US)

(21) Appl. No.: 19/245,499

(22) Filed: Jun. 23, 2025

Related U.S. Application Data
(60) Provisional application No. 63/735,340, filed on Dec. 18, 2024.

(51) Int. Cl. G16H 20/70

(52) U.S. Cl. CPC G16H 20/70

(57) ABSTRACT

The Timaci Method is a structured framework designed to cultivate emotionally aligned romantic relationships through a clear, phased approach. It starts with self-reflection exercises that help individuals identify their five-core relationship needs-viewing anything beyond those as bonuses. Early communication, including structured dialogue before and during the Kiskii Date phase, promotes emotional transparency from the start. As couples deepen their connection, they move into the Rencya Negotiation phase, aligning expectations around intimacy, finances, lifestyle, and future planning. Designed for use in coaching, education, or digital platforms, the method offers a practical blueprint for building mature, intentional partnerships. Unlike algorithm-driven dating models, the Timaci Method emphasizes self-awareness, values-based communication, and strategic compatibility. Through guided tools and purposeful interaction, it helps individuals foster authentic, lasting relationships rooted in mutual respect, growth, and emotional safety.

### FIELD OF THE INVENTION

The present invention relates to systems and methods for structured relationship development and emotional compatibility assessment. More specifically, it concerns tools that support romantic relationship formation through intentional self-reflection, transparent communication, and structured negotiation. The method can be implemented via printed materials, digital platforms, or guided sessions to enhance relationship quality, alignment, and longevity.

### BACKGROUND OF THE INVENTION

In contemporary dating culture, the landscape of romantic relationship formation has become increasingly shaped by rapid interactions, superficial engagements, and digital platforms that prioritize convenience, efficiency, and appearance-based filtering over emotional depth and authentic connection. The widespread use of dating applications and social media has facilitated unprecedented access to potential partners; however, this accessibility has not translated into higher rates of meaningful, lasting relationships. Instead, many individuals find themselves trapped in cycles of repeated relational failures characterized by poor communication, unmet emotional needs, and mismatched values. Emotional burnout, confusion about personal relational goals, and premature attachment to incompatible partners are common outcomes in this environment.

Despite the availability of numerous tools aimed at improving matchmaking and relationship success—such as dating apps, personality tests, and relationship advice content—there remains a significant lack of frameworks that support intentional, emotionally mature relationship development. Existing solutions often prioritize short-term attraction metrics, aesthetic appeal, or data-driven compatibility scores rather than fostering genuine emotional alignment, personal growth, and strategic long-term planning.

Prior inventions have focused heavily on diagnostic or algorithmic approaches. For example, U.S. Pat. No. 6,618,716 B1 discloses a system for matching individuals based on personality typing, aiming to pair partners through standardized personality profiles. U.S. Pat. No. 8,825,267 B2 describes a compatibility algorithm using personality surveys to identify potential relational matches. U.S. Pat. No. 10,987,143 B2 introduces a system for relationship coaching using mood tracking and journaling features in a digital environment. While these inventions provide valuable insights into individual preferences or emotional states, they do not sufficiently address the need for human-led, face-to-face dialogue, intentional value-sharing, or structured negotiation of relationship expectations.

Moreover, the increasing reliance on automation and data analytics in romantic matchmaking often reduces relational development to a transactional or gamified process, stripping it of the depth, vulnerability, and intentionality necessary for meaningful connection. These systems may identify surface-level compatibility but fail to equip individuals with the skills or frameworks needed to navigate the complexities of and proactive management of potential conflicts.

The Timaci Method arises in direct response to these deficiencies. It offers a comprehensive, human-centered framework that positions individuals as proactive architects of their relational journeys. By integrating elements of psychological coaching, self-reflective exercises, guided communication protocols, and structured future-oriented planning, the Timaci Method fosters emotional congruence, intentional connection, and long-term relational resilience. Its unique phases—such as the Kiskii Date for structured early-stage dialogue and the Rencya Negotiation for future planning—transform typical dating interactions into opportunities for transparency, alignment, and mutual growth.

Furthermore, the Timaci Method is designed with flexibility and scalability in mind. It is adaptable for delivery via printed workbooks, coaching sessions, group workshops, or digital applications that provide interactive tools and progress-tracking features. This versatility allows the method to serve a wide range of users, from individuals seeking self-guided relationship development to professionals offering structured relationship education. By addressing the critical gaps left by prior art and informal dating practices, the Timaci Method represents a significant advancement in the field of romantic relationship development and emotional compatibility assessment.

### SUMMARY OF THE INVENTION

The Timaci Method is a structured, multi-phase system designed to help individuals create emotionally aligned, sustainable romantic relationships. It blends introspective self-reflection, guided communication, and strategic future planning into a cohesive framework. The method can be implemented independently, with a coach, or through digital platforms.

Users begin by reflecting on the personality traits they bring to a relationship, as well as the qualities they desire in a future relationship. They examine their emotional core relational needs using tools like the “Reflect on five Top qualities” chosen from a curated list of over 50+qualities in relationship (such as best friends, congruent, loyalty, great communication, compromising and etc), they select their top five relationship needs in a relationship, taking time to deeply consider personal stories and goals related to each value. For example, 90% of individuals were unaware of what their top needs were before engaging in this reflective exercise. Those who completed the exercise reported a greater ability to find the right match from the beginning.

The Kiskii Date, named after the Japanese word for “miracle,” offers a structured format for either a first or second date. Participants share their top five needs before the Kiskii Date and use guided questions to explore their alignment, such as, “What are the five most important qualities you seek in a healthy relationship?” “What life experiences have shaped these five needs?” and “What do you consider essential for maintaining daily respect in treatment in your ideal relationship?” This process encourages emotional transparency and helps identify compatibility early on.

If the Kiskii Date reveals promising alignment, couples proceed to Rencya Negotiation—a structured dialogue covering seven key relational domains: schedules, intimacy, finances, parenting, lifestyle, social integration, and contingency planning. Worksheets and scripts help partners express expectations, non-negotiable, and compromises, creating a shared, revisable relational agreement. This method differs from algorithmic matchmaking or personality-typing systems by emphasizing human-led, in-person dialogues and strategic negotiation. Optional digital tools include compatibility tests, boundary-setting guides, and post-negotiation check-ins. The Timaci Method thus provides an actionable blueprint for developing intentional, first emotionally intelligent partnerships.

### DESCRIPTION OF FIGURES

FIG. 1 illustrates the eight-step Timaci Method, including Self-Reflection (10), Manifesting (12), Profile Preparation (14), Communication Before Kiskii Date (16), Kiskii Date (18), Rencya Negotiation (20), Happily Ever After (22), and Timaci Beliefs (24).

FIG. 2 depicts the Kiskii Date framework, which includes Pre-Date Preparation (30), Values Discussion (32), Emotional Safety (34), and Home Chemistry Check (36), arranged in a structured circular sequence.

FIG. 3 shows the Rencya Negotiation process (20), centered around seven key relational domains: Schedules (40), Intimacy (42), Finances (44), Parenting (46), Lifestyle (48), Social Integration (50), and Contingency Planning (52).

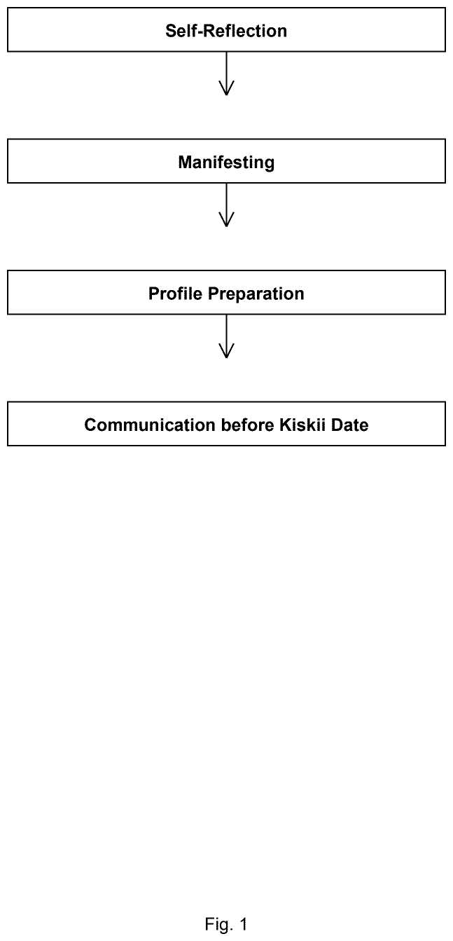

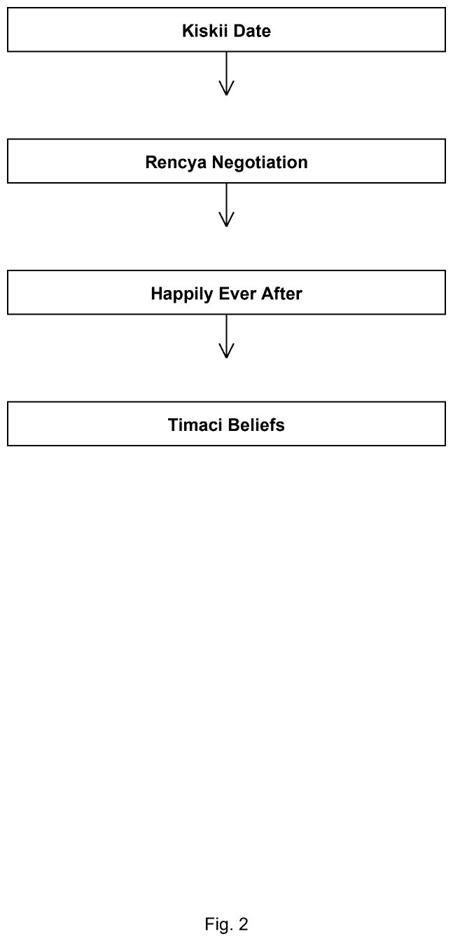

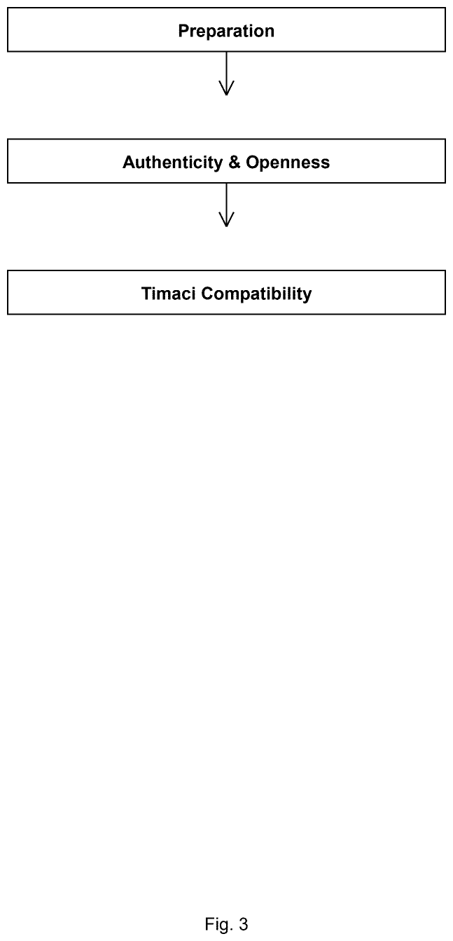

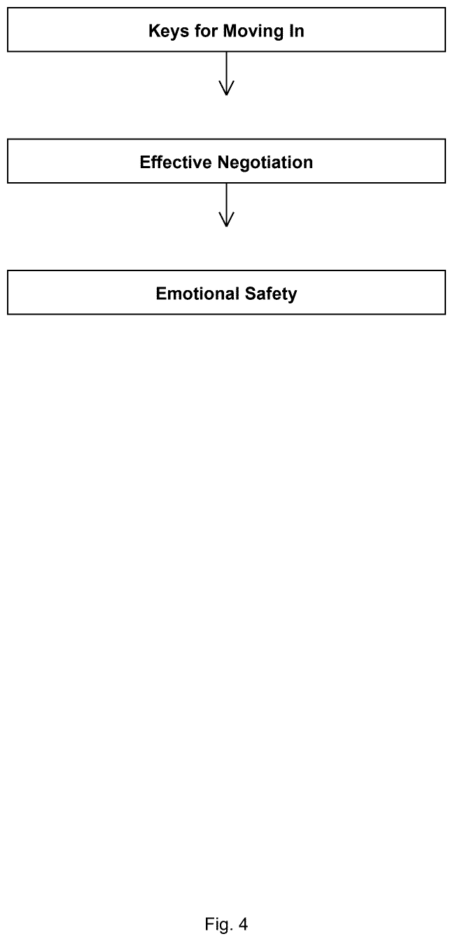

### DETAILED DESCRIPTION OF INVENTION

This application claims the benefit of U.S. Provisional Ser. No. 63/735,340, filed on Dec. 18, 2024, the entire contents of which are hereby incorporated by reference. The Timaci Method is a structured, multi-phase relationship development system designed to foster emotionally mature, intentional romantic connections. By integrating psychological insight, practical communication tools, and value-driven exercises, it supports individuals and couples in building authentic, lasting relationships. The method unfolds across three overarching phases—Self-Reflection, Kiskii Date, and Rencya Negotiation—which are further divided into eight detailed stages. Each stage functions as a stepping stone to the next, promoting deep internal alignment, conscious partner selection, and sustainable relationship dynamics.

The journey begins with Self-Reflection, a foundational phase that prompts users to explore their core values, emotional patterns, past relationship dynamics, and future aspirations. Through structured tools such as the Relationship Audit Worksheet and Personal Values Map, individuals identify their emotional triggers and articulate their five non-negotiable values. A curated list of over 50 relational values is provided to guide users in selecting and internalizing their top priorities. This introspective work is not only aimed at healing from past experiences but also at gaining clarity and confidence before re-entering the relational sphere. The emphasis on internal clarity ensures that users engage in future relationships from a position of self-awareness and authenticity rather than unresolved emotional patterns or reactive behaviors.

The second stage, Manifesting, builds on this internal work by guiding users to envision and intentionally attract relationships that align with their core values. Techniques such as the Manifestation Vision Board and Daily Intention Tracker are employed to help users declare their emotional goals and cultivate an abundant, non-scarcity mindset. Rather than fixating on surface-level traits, individuals are encouraged to articulate the emotional, psychological, and lifestyle qualities they wish to experience in a partner and relationship. This practice refines the user's ability to recognize and attract aligned connections, increasing the likelihood of meaningful, value-based partnerships.

Once clarity and intention have been established, the Profile Preparation stage translates inner insights into an authentic outward presentation. Whether preparing a dating profile or entering a new relational space, users are guided through templates that help craft narratives emphasizing personal growth, relational purpose, and the five identified non-negotiables. This approach ensures coherence between internal values and external presentation, reducing the likelihood of mismatches and enabling potential partners to connect with the user's true self.

Before any in-person engagement, the Communication Before Kiskii Date stage equips users with intentional dialogue tools. Templates and guides are provided to help express needs clearly, establish boundaries, and create an emotionally safe conversational environment. These early communications are grounded in the principles of non-judgmental listening and mutual understanding. By front-loading crucial value-based conversations, this phase reduces misalignment and sets the tone for emotionally intelligent interaction.

The Kiskii Date phase introduces a unique, structured approach to first meetings. Unlike conventional dating models, the Kiskii Date centers on emotional safety, openness, and intentional value exchange. Prior to the date, participants prepare by clearly identifying and practicing how to express their top five relational needs. During the date, dialogue templates guide conversations around each value, encouraging discussion of its origin, day-to-day importance, and what it looks like in practice. Emotional safety protocols—such as active listening and vulnerability framing—are emphasized to create a trustworthy environment. Post-date reflection worksheets allow participants to evaluate the presence of “home chemistry,” emotional resonance, and compatibility, thus avoiding premature emotional attachment and encouraging data-driven relational decisions.

Following promising Kiskii Dates, the process advances to Rencya Negotiation, a multi-layered cohabitation planning phase. This stage is rooted in emotional transparency and structured compromise. Couples engage in deep conversations across seven key relational domains: schedules and routines, intimacy and affection, financial management, parenting and future planning, lifestyle preferences, social integration (family and friends), and contingency planning for challenges or crises. Each domain is explored using the Relationship Alignment Sheet, which prompts both partners to record agreements, divergences, and unresolved areas for future dialogue. Guided scripts and prompts support emotional regulation and reduce defensiveness during negotiations, allowing both individuals to express expectations while honoring the other's needs. This proactive framework mitigates common cohabitation conflicts and creates a personalized blueprint for shared life success.

The Happily Ever After phase focuses on long-term relationship maintenance. Couples are supported in revisiting and recalibrating their values and agreements through tools like the Monthly Relationship Health Wheel, Shared Goal Worksheets, and Emotional Wellness Assessments. These resources encourage partners to view relational health as an evolving process rather than a static achievement. The emphasis on communication, emotional growth, and mutual accountability nurtures lasting intimacy and adaptability over time.

The final stage, Timaci Beliefs, promotes the integration of learned practices into lifelong relational habits. Participants are encouraged to reflect on their journey, mentor others, and re-engage with the method through periodic self-audits. This stage emphasizes love as a dynamic, evolving partnership grounded in authenticity, trust, and continuous growth. Participants are invited to share insights with their broader communities, thereby amplifying the ripple effect of conscious relationships and

What sets the Timaci Method apart is not only its structured content but also its versatile delivery mechanisms. It can be implemented through physical formats like printed workbooks or used in coaching programs and in-person workshops. More innovatively, the method is optimized for digital platforms, offering interactive tools such as real-time dialogue aids, reflective journaling modules, boundary-setting guides, and relationship progress trackers. These features enhance accessibility and engagement, making the method suitable for individuals navigating dating, committed couples, and professionals such as therapists and relationship coaches. Optional modules—like compatibility assessments, video walkthroughs, and milestone reminders—offer further personalization and support.

Ultimately, the Timaci Method represents a paradigm shift in relationship development, moving away from trial-and-error dating and toward intentional, values-driven partnership building. By guiding users from self-understanding to sustainable relational design, it empowers individuals to become active architects of their romantic lives. Through its emphasis on emotional clarity, structured communication, and mutual respect, the Timaci Method fosters partnerships built not just on attraction, but on deep compatibility, shared purpose, and resilient love.

1. A method for facilitating emotionally aligned romantic relationships, the method comprising conducting a self-reflection process to identify relational values of an individual, wherein the self-reflection includes structured exercises for assessing emotional patterns, relational history, and personal priorities; selecting core relationship needs based on the self-reflection from a predefined list of relational attributes representing emotional and practical values; participating in a structured first-date dialogue referred to as a Kiskii Date, wherein participants exchange the core relationship needs using guided prompts configured to promote emotional transparency; conducting a post-date assessment in which participants reflect upon emotional resonance, value alignment, and relational compatibility; and engaging in a structured negotiation process referred to as Rencya negotiation, the process addressing relational domains including daily schedules, intimacy, financial planning, parenting intentions, lifestyle preferences, social circle integration, and contingency plans.
1.1. The method of claim 1, wherein the self-reflection includes journaling exercises and value mapping diagrams configured to facilitate internalization and prioritization of relational needs.
1.2. The method of claim 1, wherein the Kiskii Date includes a dialogue template with guided prompts and questions configured to ensure emotional transparency, encourage storytelling, and support mutual understanding.
1.3. The method of claim 1, wherein the Rencya negotiation includes guided conversations and documentation tools covering at least schedules, intimacy, finances, parenting, lifestyle preferences, social relationships, and contingency planning.
1.4. The method of claim 1, further comprising a post-date reflection tool including worksheets or digital interfaces configured to assist participants in evaluating emotional connection and potential compatibility.
1.5. The method of claim 1, wherein the method is implemented using a digital platform or print-based materials including at least one of workbooks, mobile applications, or online portals configured to provide tools for each stage of the method.

2. A system for facilitating structured romantic relationship development, the system comprising a self-assessment interface configured to guide users through self-reflection and value identification processes; a guided communication module configured for use during initial dates, the module including structured dialogue templates and emotional transparency prompts; a relational alignment tool configured for future planning, the tool comprising negotiation frameworks for relational domains including lifestyle, intimacy, and finances; and a storage component configured to save and allow users to revisit relational agreements, reflections, and progress records over time.
2.1. The system of claim 2, wherein the system includes a digital interface integrated with real-time communication aids for use during dates and negotiations.
2.2. The system of claim 2, wherein the system includes tools for tracking relational growth goals, the tools including goal-setting modules and periodic review prompts.
2.3. The system of claim 2, wherein the system includes compatibility testing modules configured to assess alignment across emotional, practical, and value-based dimensions.
2.4. The system of claim 2, wherein the system is configured to facilitate periodic relational health assessments through questionnaires, check-ins, and progress dashboards.
2.5. The system of claim 2, wherein the system includes functionality for storage, retrieval, and revision of relational agreements, negotiation outcomes, and personal reflections, enabling ongoing adaptation and refinement of relationship plans.
